(19) United States
(12) Patent Application Publication
(10) Pub. No.: US 2006/0016686 A1
(43) Pub. Date: Jan. 26, 2006

(54) Blood glucose meter

(76) Inventor: Chun-Yu Chen, Taipei (TW)

Correspondence Address: BRUCE H. TROXELL, SUITE 1404, 5205 LEESBURG PIKE, FALLS CHURCH, VA, 22041, US

(21) Appl. No.: 11/088,790

(22) Filed: Mar. 25, 2005

(30) Foreign Application Priority Data
Jul. 23, 2004 (TW) 93211643

(51) Int. Cl. G01N 33/487; G01N 27/26

(52) U.S. Cl. 204/403.01; 204/403.14

(57) ABSTRACT

A blood glucose meter. The blood glucose meter comprises an input interface in which an inspection card having samples of a patient is inserted outputting a current signal, a current/voltage converting circuit coupled to the input interface and converting the current signal into an analog voltage signal, an analog/digital converter coupled to the current/voltage converting circuit and converting the analog voltage signal into a digital voltage signal, a transmit interface coupled to an external data recognizer coupled to a computer interface through which a control signal is transmitted, thereby updated data are input into the transmit interface, a compensation unit receiving the updated data and updating data therein, a microprocessor determining inspection mode for the inspection according to the updated data in the compensation unit and outputting the inspection result, and a memory unit recording the result.

### 1. FIELD OF THE INVENTION

The present invention relates to a blood glucose meter, and more particularly, to a blood glucose meter capable of receiving an inspection card comprising a plurality of enzymes reacting with a patient's blood, and processing the inspection card with a current/voltage converting circuit and a microprocessor for showing the result on a display as patient's record.

### 2. BACKGROUND OF THE INVENTION

Following the advance of medical technology, most diseases can be cured or prevented enabling people, male or female, have a longer life than ever. Nowadays, people can do self-inspection by means of some scientific inspection methods and provide data of inspection for doctors to treat the diseases properly.

Diabetes is one of the modem diseases, which is caused by the pancreas unable to secrete insulin (or insufficient insulin). Insulin decomposes glucose in human's blood and transfers it into useful energy. The diabetes patient cannot effectively decompose glucose such that the glucose is accumulated in blood increasing the probability of apoplexy, myocardial infarction, blindness and kidney exhaustion. Therefore, the diabetes patient has to take medicine regularly and go to hospital for follow-up treatment to prevent related complications.

By the help of modem electrical instruments, patients can now perform glucose measurement and record data in their own house, thereby proving the data for doctors as a reference while taking medical treatments. In addition, the diabetes patient keeps track of their glucose data for controlling their diet so as to avoid any complications. The invention is to provide a simple glucose measuring electronic device for helping diabetes control.

### SUMMARY OF THE INVENTION

It is an primary object of the invention to provide a blood glucose meter capable of receiving an inspection card comprising a plurality of enzymes reacting with a patient's blood, and processing the inspection card with a current/voltage converting circuit and a microprocessor for showing the result on a display as patient's record.

Another object of the invention is to provide data recognizer or a data updating device adapted for updating data in a blood glucose, and thus enabling the blood glucose meter to be able to perform various inspections.

To achieve the above objects, the blood glucose meter of the invention comprises:
an input interface, for receiving an inspection card of samples and outputting a current;
a current/voltage converting circuit, coupled to the input interface for converting the outputted current into an analog voltage;
an analog/digital converter, coupled to the current/voltage converting circuit for converting the analog voltage signal into a digital voltage signal;
a transmit interface, coupled to an external data recognizer which is further coupled to a computer interface, wherein an updated data is transmitted into the transmit interface while a control signal is issued through the computer interface;
a compensation unit, receiving the updated data and updating data therein;
a microprocessor, selecting an inspection mode for performing an inspection basing on the updated of the compensation unit, and outputting an inspection result; and
a memory unit, for recording the inspection result.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a block diagram of a blood glucose meter of the invention.

FIG. 2 is a block diagram of a data updating device of the invention.

FIG. 3 is a schematic diagram of an array of at least three resistance values according to the invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

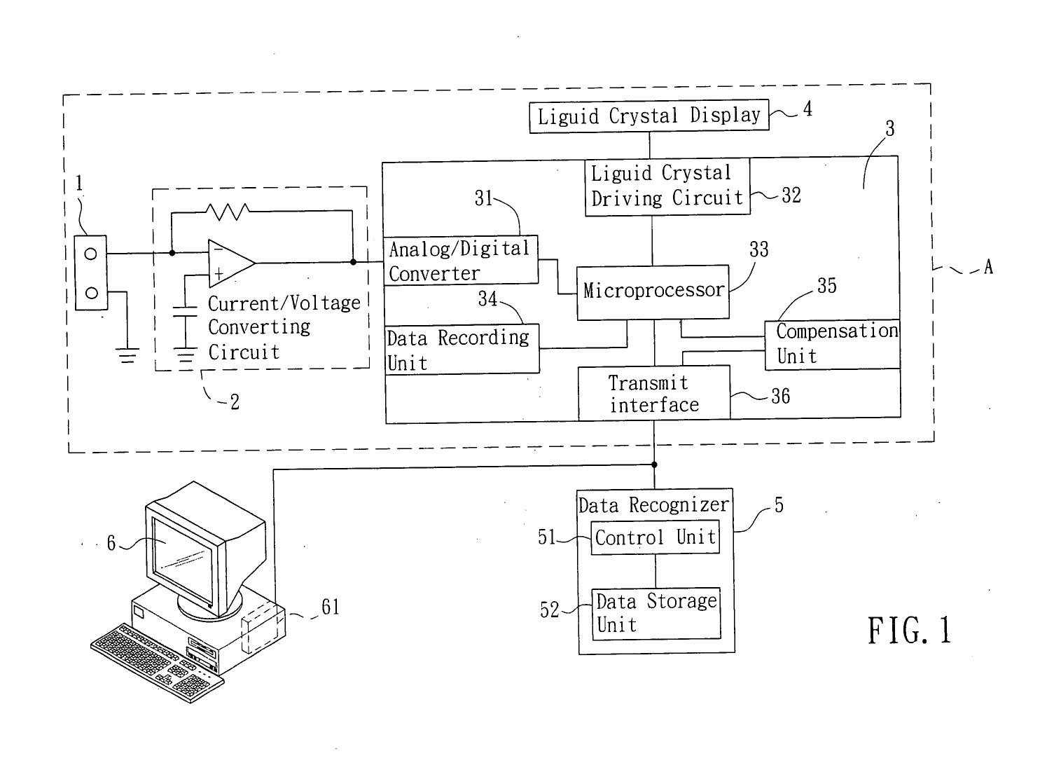

Referring to FIG. 1, a blood glucose meter A comprises: an input interface 1, for receiving an inspection card of samples where the samples react with the enzymes therein so as to generate a small current to be outputted from the input interface 1; a current/voltage converting circuit 2, receives the small current and converts the same into a voltage to be outputted from the current/voltage converting circuit 2; a processing unit 3, for receiving the voltage from the current/voltage converting circuit 2 and processing the same, the processing unit 3 further comprising: an analog/digital converter 31, a liquid crystal driving. circuit 32, a microprocessor 33, a data recording unit 34, a compensation unit 35 and a transmit interface 36, wherein:
the analog/digital converter 31 is coupled to the current/voltage converting circuit 2 for converting the analog voltage signal into a digital voltage signal;
the transmit interface 36 is coupled to an external data recognizer 5 which is further coupled to a computer interface 61 of a computer 6, such that an updated data is transmitted into the transmit interface 36 while a control signal is issued through the computer interface 61;
the compensation unit 35 is capable of receiving the updated data and updating data resided therein.
the microprocessor 33 is used for selecting an inspection mode for performing an inspection basing on the updated of the compensation unit 35, and outputting an inspection result;
the memory unit 34 is used for recording the processed result of the microprocessor 33.
the liquid crystal driving circuit 32 is used for driving a liquid crystal display 4 to show the inspection result.

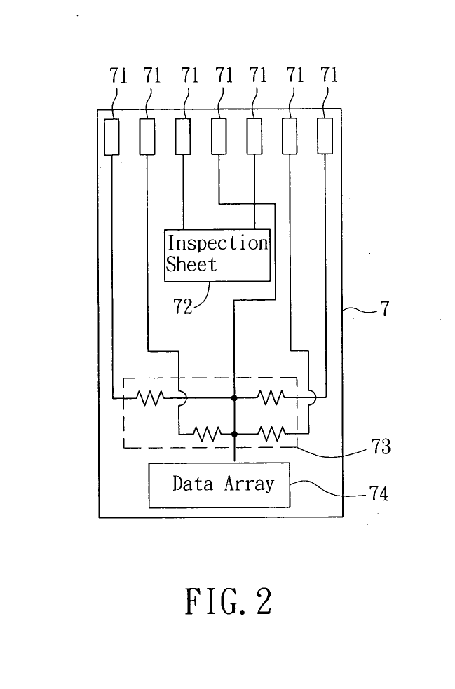

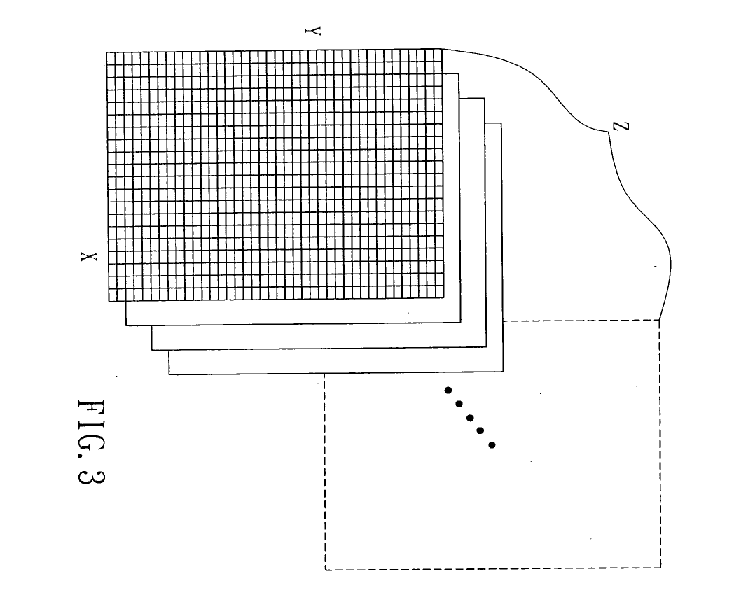

Referring to FIGS. 2 and 3, a data updating device 7 includes a plurality of electrical contacts 71 connected to an inspection sheet 72, a resistance array 73 , and a data array 74 in respective. The resistance array 73 is substantially an array consisted of at least two resistors. If the resistance array 73 is consisted of more than three resistors, the corresponding X, Y and Z values formed of the resistance array 73 can map with the data array 74 of chaotic codes, which has a broader error tolerance to avoid data mis-selection. When an inspection mode is selected, the inspection mode of the compensation unit 35 in FIG. 1 is updated accordingly and the microprocessor 33 will then execute an inspection according to the selected inspection mode. The data updating device 7 of the invention can replace the transmit interface 36 and the external recognizer 5 to update the inspection mode of the compensation unit 35.

A broadcast unit (not shown) can be added to the blood glucose meter, and a speaker or a headset can be used to broadcast the inspection result. The broadcast unit is an integrated chip in which human voice is recorded. The broadcast unit is for old patients who have poor eyesight, and it is more user friendly. The broadcasting not only informs inspection result, but also informs the current inspection mode and residue battery power, etc.

In FIGS. 1 to 2, the invention employs a data recognizer or a data updating device to update data in a blood glucose meter so that the blood glucose meter can be used for multiple measurement purposes. The invention can also be used in the field of biology inspection and manufacturing bio-chip and the like.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A-blood glucose meter, comprising:
an input interface, for receiving an inspection card of samples and outputting a current;
a current/voltage converting circuit, coupled to the input interface for converting the current into an analog voltage signal;
an analog/digital converter, coupled to the current/voltage converting circuit for converting the analog voltage signal into a digital voltage signal;
a transmit interface, coupled to an external data recognizer which is further coupled to a computer interface, wherein an updated data is transmitted into the transmit interface while a control signal is issued through the computer interface;
a compensation unit, for receiving the updated data and updating data therein;
a microprocessor, selecting an inspection mode for performing an inspection basing on the updated of the compensation unit, and outputting an inspection result; and
a memory unit, for recording the inspection result.

2. The blood glucose meter as claimed in claim 1, wherein the meter further comprises a display unit for displaying the result stored in the memory unit.

3. The blood glucose meter as claimed in claim 2, wherein the display unit is a liquid crystal display.

4. The blood glucose meter as claimed in claim 2, wherein the display unit is a matrix panel composing of a plurality of light emitting diodes.

5. The blood glucose meter as claimed in claim 1, wherein the meter further comprises a broadcast unit, capable of using at least an object selected from the group consisting of a speaker and a headset for a voice broadcasting.

6. The blood glucose meter as claimed in claim 5, wherein the broadcast unit further comprises an integrated circuit in which human voice is recorded.

7. The blood glucose meter as claimed in claim 1, wherein the data update device comprises an electrical contact, an inspection sheet, a resistance array and a data array.

8. The blood glucose meter as claimed in claim 1, wherein a plurality of enzymes are resided in the inspection card for reacting with the patient's blood to create a current.

9. A blood glucose meter, comprising:
an input interface, for receiving an inspection card of samples and outputting a current;
a current/voltage converting circuit, coupled to the input interface for converting the current into an analog voltage signal;
an analog/digital converter, coupled to the current/voltage converting circuit for converting the analog voltage signal into a digital voltage signal;
a compensation unit, for receiving the updated data and updating data therein;
a microprocessor, selecting an inspection mode for performing an inspection basing on the updated of the compensation unit, and outputting an inspection result;
a memory unit, for recording the inspection result; and
an update interface, for receiving a data update device to compensate the inspection level of the compensation unit.

10. The blood glucose meter as claimed in claim 9, wherein the data update device compensate the compensation unit according to values in one of the at least two resistance arrays of the data update device.

11. The blood glucose meter as claimed in claim 10, wherein the meter further comprises a display unit for displaying the result stored in the memory unit.

12. The blood glucose meter as claimed in claim 11, wherein the display unit is a liquid crystal display.

13. The blood glucose meter as claimed in claim 11, wherein the display unit is a matrix panel composing of a plurality of light emitting diodes.

14. The blood glucose meter as claimed in claim 9, wherein the meter further comprises a broadcast unit, capable of using at least an object selected from the group. consisting of a speaker and a headset for a voice broadcasting.

15. The blood glucose meter as claimed in claim 14, wherein the broadcast unit further comprises an integrated circuit in which human voice is recorded.

16. The blood glucose meter as claimed in claim 9, wherein a plurality of enzymes are resided in the inspection card for reacting with the patient's blood to create a current.
